(19) United States
(12) Plant Patent Application Publication
(10) Pub. No.: US 2007/0079405 P1
(43) Pub. Date: Apr. 5, 2007

(54) Variety of raspberry named 'Rafzaqu'

(76) Inventor: Peter Hauenstein, Wasterkingen (CH)

Correspondence Address: CHRISTIE, PARKER & HALE, LLP, PO BOX 7068, PASADENA, CA, 91109-7068, US

(21) Appl. No.: 11/241,290

(22) Filed: Sep. 30, 2005

(51) Int. Cl. A01H 5/00

(52) U.S. Cl. PLT/204

(57) ABSTRACT

A new and distinct variety of raspberry plant, designated ‘RAFZAQU’, characterized by large, firm, easily detachable fruit with excellent flavor, medium spine density on very young shoots, free to touching lateral leaflets and a medium time of ripening on canes.

### CLASSIFICATION

The variety is botanically known as Rubus idaeus.

### VARIETAL DENOMINATION

The new raspberry plant has the varietal name of ‘RAFZAQU’.

### SUMMARY OF THE INVENTION

The present invention relates to a new and distinct variety of raspberry plant designated as ‘RAFZAQU’. This new variety is a result of a controlled cross between the fall raspberry variety ‘AUTUMN BLISS’ (U.S. Plant Pat. No. 6,597) and the summer raspberry variety ‘RAFZETER’ (not patented in the United States; sold as HIMBO QUEEN).

The new variety is a fall variety, having strong growth, producing fewer runners (stolons) than ‘AUTUMN BLISS’, and having similar resistance to Phytophtora as ‘AUTUMN BLISS’. The fruit of the new variety is very large compared to most known commercial varieties, on the order of six to 8 grams, and is firm, easy to transport, brilliant red in color, without darkening after harvest, conical in shape, and easily detachable with excellent flavor. Fruit of the new variety can be harvested in mid-Switzerland from about August 20 (i.e., approximately 2 weeks after ‘AUTUMN BLISS’), for a duration of 6 to 7 weeks.

After its selection, the new variety was asexually propagated by cuttings and extensively tested in fruiting fields. This propagation has demonstrated that the combination of traits disclosed herein as characterizing the new variety are fixed and remain true to type through successive generations of asexual reproduction.

### Comparison to Closest Commercial Cultivar

The new variety differs from the commercial cultivar ‘AUTUMN BLISS’ as follows:

| | ‘AUTUMN BLISS’ | ‘RAFZAQU’ |
|---|---|---|
| Young shoot - density of | Dense to very close | Medium |
| spines in central third | | |
| Leaf - overlapping of | Touching to overlapping | Free to touching |
| leaflets | | |
| Time of ripening on canes | Very early to early | Medium |

### BRIEF DESCRIPTION OF THE DRAWINGS

The accompanying color photographs show a typical specimen of the new variety as nearly true as it is possible to make in color reproductions.

FIG. 1 is a photograph of the leaves and fruit of the new variety.

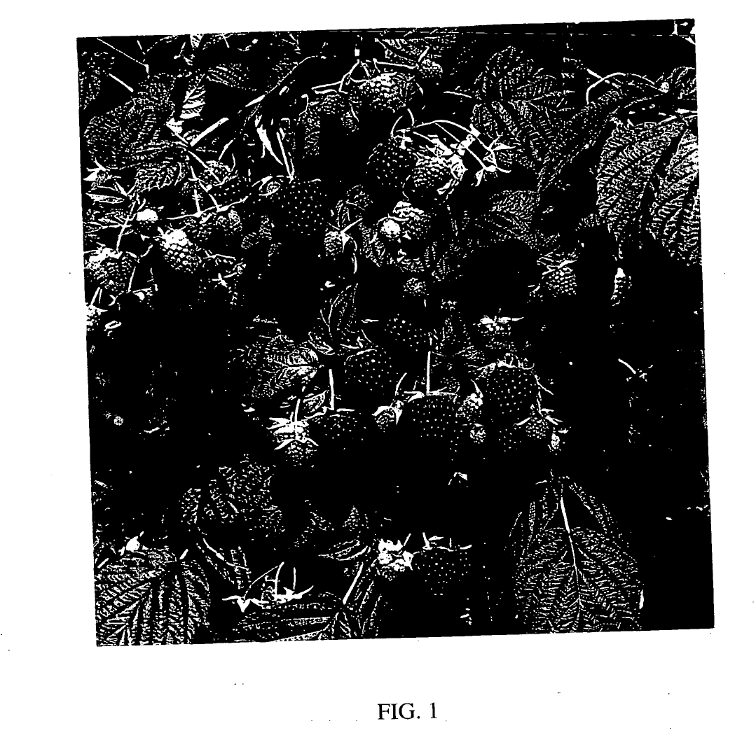

FIG. 2 is a close-up photograph of fruit of the new variety.

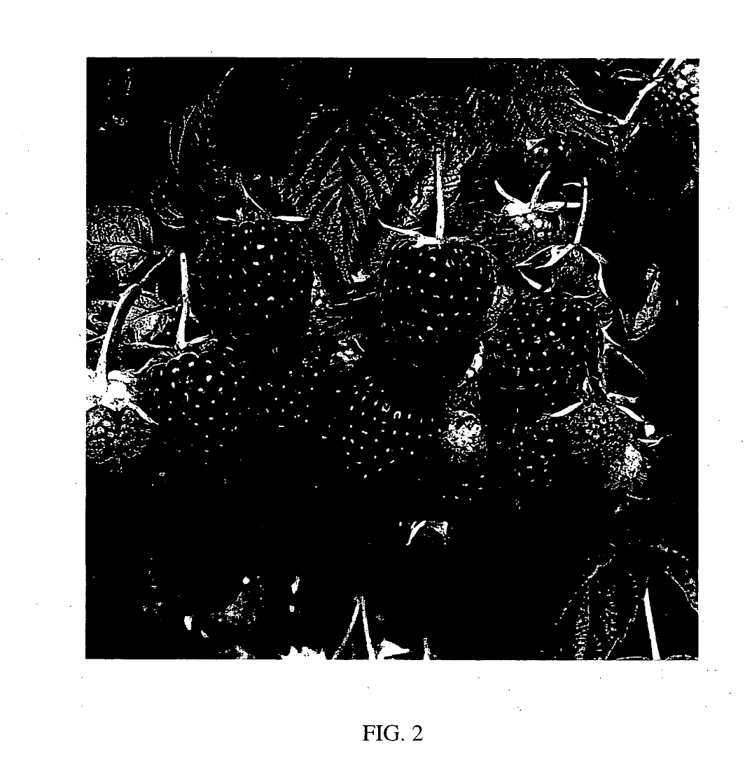

### DETAILED DESCRIPTION OF THE INVENTION

The phenotypical descriptions, measurements and color designations stated for the new variety ‘RAFZAQU’ may vary, depending upon variations in environmental factors, including weather (temperature, humidity and light intensity), day length, soil type, location and cultural conditions. ‘RAFZAQU’ has not been observed under all possible environmental conditions.

| | |
|---|---|
| Anthocyanin coloration of very | Present |
| young shoots | |
| Intensity of anthocyanin coloration | Weak to medium |
| of very young shoots | |
| Number of young shoots | Medium |
| Spines on young shoots | Present |
| Density of spines in central third of | Medium |
| young shoot | |
| Color of spines on young shoots | Purple |
| Bloom on full grown shoot | Medium to strong |
| Cane - length | Long |
| Leaf - relief between the veins | Medium to strong |
| Leaf - number of leaflets | Sometimes three, sometimes five |
| Leaf - overlapping of lateral leaflets | Free to touching |
| Leaf - color of upperside | Medium to dark green |
| Lateral leaflet - length of stalklet | Very short |
| (lower pair) | |
| Flower - size | Large |
| Flower - anthocyanin coloration of | Present |
| pedicel | |
| Flower - intensity of anthocyanin | Medium to strong |
| coloration of pedicel | |
| Fruit - color | Medium red |
| Fruit - size | Medium to large |
| Fruit - length:width ratio | Longer than broad |
| Fruit - firmness | Medium |
| Fruit - glossiness | Medium to strong |
| Fruit - adherence to plug | Medium |
| Season of fruiting | Autumn |
| Time of shoot emergence | Early |
| Time of ripening on canes | Medium |
| Length of harvest | Medium to long |

What is claimed is:

1. A new and distinct variety of Rubus idaeus plant, identified as ‘RAFZAQU’, substantially as shown and described.
